(19) United States
(12) Patent Application Publication
(10) Pub. No.: US 2012/0265105 A1
(43) Pub. Date: Oct. 18, 2012

(54) ULTRASOUND CONSTIPATION RELIEF DEVICE

(76) Inventor: Masahito Tsutsui, Komae-shi (JP)

(21) Appl. No.: 13/516,531

(22) PCT Filed: May 24, 2011
(86) PCT No.: PCT/JP2011/061867

§ 371 Date: Jun. 15, 2012

(30) Foreign Application Priority Data
May 28, 2010 (JP) 2010-122633

(51) Int. Cl. A61N 7/00

(52) U.S. Cl. 601/2

(57) ABSTRACT

A therapy device capable of relieving constipation by applying stimulation from a back to a lumbar spine and a vicinity thereof, the device includes: an ultrasound signal oscillation circuit; and a head, which is placeable from the back on at least a region between a third lumbar spine and a fourth lumbar spine, a region between the fourth lumbar spine and a fifth lumbar spine, or any one or two or more of the third lumbar spine, the fourth lumbar spine, and the fifth lumbar spine. The head includes a piezoelectric element, which generates ultrasound capable of being transmitted from the back to the lumbar spine or a region between lumbar spines, and the head is sized to be placeable on the region between the lumbar spines, and is capable of outputting ultrasound vibration having frequency and power capable of stimulating a pressure point or nerve relating to constipation.

### TECHNICAL FIELD

The present invention relates to a constipation relief device having a function of constipation relief (including defecation induction) by ultrasound, and to a composite type constipation relief device having both a constipation relief function and a high-frequency electric potential therapy function.

### BACKGROUND ART

Since ancient times, many people have suffered from constipation. There are patent applications on a constipation improver (Patent Literature 1) and a defecation inducer (Patent Literature 2), but a therapy device effective for constipation relief has not been developed yet.

### CITATION LIST

### Patent Literature

[PTL 1]: JP 2004-75601 A

[PTL 2]: JP 2008-13582 A

### SUMMARY OF INVENTION

### Technical Problem

Both the constipation improver of Patent Literature 1 and the defecation inducer of Patent Literature 2 are oral agents, and hence some persons who take the agent feel constitutionally uncomfortable.

The problem to be solved by the present invention is to provide a device capable of relieving constipation or inducing defecation by being used to be placed on a predetermined external site of a human body.

### Solution to Problem

An ultrasound constipation relief device according to the present invention includes: an ultrasound signal oscillation circuit; a single head, which is placeable from outside the back on at least a region between a third lumbar spine and a fourth lumbar spine, a region between the fourth lumbar spine and a fifth lumbar spine, or any one or two or more of the third lumbar spine, the fourth lumbar spine, and the fifth lumbar spine (hereinafter, those parts are referred to as “lumbar spines L₃ to L₅”); and a piezoelectric element, which is to be vibrated by an ultrasound signal oscillated from the ultrasound signal oscillation circuit to generate ultrasound capable of being transmitted from outside the back to the lumbar spine or a region between lumbar spines, in which: the piezoelectric element is fixed onto the single head made of a hard material which easily transmits ultrasound vibration generated from the piezoelectric element; the piezoelectric element is capable of generating ultrasound vibration having a frequency of 1.5 MHz to 2.0 MHz; the single head has a size which is placeable on the region between the third lumbar spine and the fourth lumbar spine, the region between the fourth lumbar spine and the fifth lumbar spine, or the any one or two or more of the third lumbar spine, the fourth lumbar spine, and the fifth lumbar spine; and the single head is capable of outputting the ultrasound vibration having the frequency and output power capable of stimulating a pressure point and a nerve relating to constipation.

An ultrasound constipation relief device according to the present invention includes: an ultrasound signal oscillation circuit; two or more heads, which are placeable from the back on at least a region between a third lumbar spine and a fourth lumbar spine, a region between the fourth lumbar spine and a fifth lumbar spine, or any one or two or more of the third lumbar spine, the fourth lumbar spine, and the fifth lumbar spine; and a piezoelectric element, which is to be vibrated by an ultrasound signal oscillated from the ultrasound signal oscillation circuit to generate ultrasound capable of being transmitted from the back to the lumbar spine or a region between lumbar spines, in which: the piezoelectric element is provided to each of the two or more heads; the piezoelectric element is capable of generating ultrasound having a frequency of 1.5 MHz to 2.0 MHz; the two or more heads are arrayed at an interval in a line or substantially in a line; the interval of the two or more heads is set so that the two or more heads are placeable on the region between the third lumbar spine and the fourth lumbar spine, the region between the fourth lumbar spine and the fifth lumbar spine, or the any two or more of the third lumbar spine, the fourth lumbar spine, and the fifth lumbar spine; and the two or more heads are made of a hard material which easily transmits ultrasound vibration of the piezoelectric element.

In the ultrasound constipation relief device of the present invention, it is desired that the piezoelectric elements of the two or more heads, which are provided at an interval, generate the ultrasound simultaneously or alternately.

In the ultrasound constipation relief device of the present invention, it is desired that the output power of the ultrasound generated from the piezoelectric element be 100 mW to 800 mW.

The ultrasound constipation relief device of the present invention further includes: a high-voltage generation portion; and an electric potential energization electrode, which is supplied with a high voltage from the high-voltage generation portion, in which the electric potential energization electrode is capable of applying the high voltage to a human body so that a microcurrent flows through the human body. Further, in the ultrasound constipation relief device of the present invention, the electric potential energization electrode is provided inside a head case of the ultrasound constipation relief device.

### Advantageous Effects of Invention

The ultrasound constipation relief device according to the present invention provides the following effects.

(1) With the head placed on a location corresponding to the lumbar spine or a region between the lumbar spines from the back, through the supply of an ultrasound signal to the piezoelectric element, the ultrasound generated from the piezoelectric element of the head is transmitted to the lumbar spine, thereby relieving constipation (facilitating bowel movement or defecation). This relief mechanism has not been medically clarified, but it is considered that constipation is relieved by stimulation to a pressure point or a nerve relating to constipation, which is located at the lumbar spine.

(2) The single head has a size which is placeable on the region between the third lumbar spine and the fourth lumbar spine, the region between the fourth lumbar spine and the fifth lumbar spine, or any one or two or more of the third lumbar spine, the fourth lumbar spine, and the fifth lumbar spine. It is therefore easy to place the head on such site, and the ultrasound generated from the piezoelectric element is reliably transmitted to the lumbar spine, thereby relieving constipation.

(3) The constipation relieving effect is conspicuous when the head is placed in use on a location corresponding to the region between the lumbar spines L₃ and L₄, the region between the lumbar spines L₄ and L₅, or the vicinity of the lumbar spines L₃ to L₅.

(4) The interval between the two or more heads is set to be the same or substantially the same as the region between the lumbar spines. Therefore, the ultrasound generated from any of the heads is reliably transmitted to the lumbar spine and the region between the lumbar spines, thereby relieving constipation.

(5) In the case of an ultrasound constipation relief device combined with an electric potential therapy device, in addition to constipation relief by ultrasound, electric potential therapy is also performed by placing the electric potential energization (application) electrode on the back and applying a high voltage generated from the high-voltage generation portion to the back via the electric potential energization electrode so as to cause a microcurrent to flow through a body.

(6) The constipation relief and the electric potential therapy can be performed simultaneously and independently, for different uses as appropriate.

(7) The piezoelectric element is fixed onto the head made of a hard material which easily transmits the ultrasound vibration, and hence the ultrasound vibration can be transmitted even from the back.

(8) The frequency of the ultrasound vibration generated from the piezoelectric element is 1.5 MHz to 2.0 MHz, and hence the constipation relieving effect is conspicuous as confirmed by an experiment to be described below.

### BRIEF DESCRIPTION OF DRAWINGS

FIG. 1 A block diagram of an example of an ultrasound constipation relief device according to the present invention.

FIG. 2 Views illustrating an example of the ultrasound constipation relief device according to the present invention, in which FIG. 2(a) is a perspective view; FIG. 2(b), a vertical cross-sectional view; FIG. 2(c), a bottom view in a state in which a rear lid is detached; FIG. 2(d), a bottom view; FIG. 2(e), a plan view; FIG. 2(f), a left side view; FIG. 2(g), a right side view; and FIG. 2(h), a front view.

FIG. 3 An appearance view illustrating an example of a device main body of the ultrasound constipation relief device according to the present invention.

FIG. 4 An appearance view illustrating an example of the usage of the ultrasound constipation relief device according to the present invention.

### DESCRIPTION OF EMBODIMENT

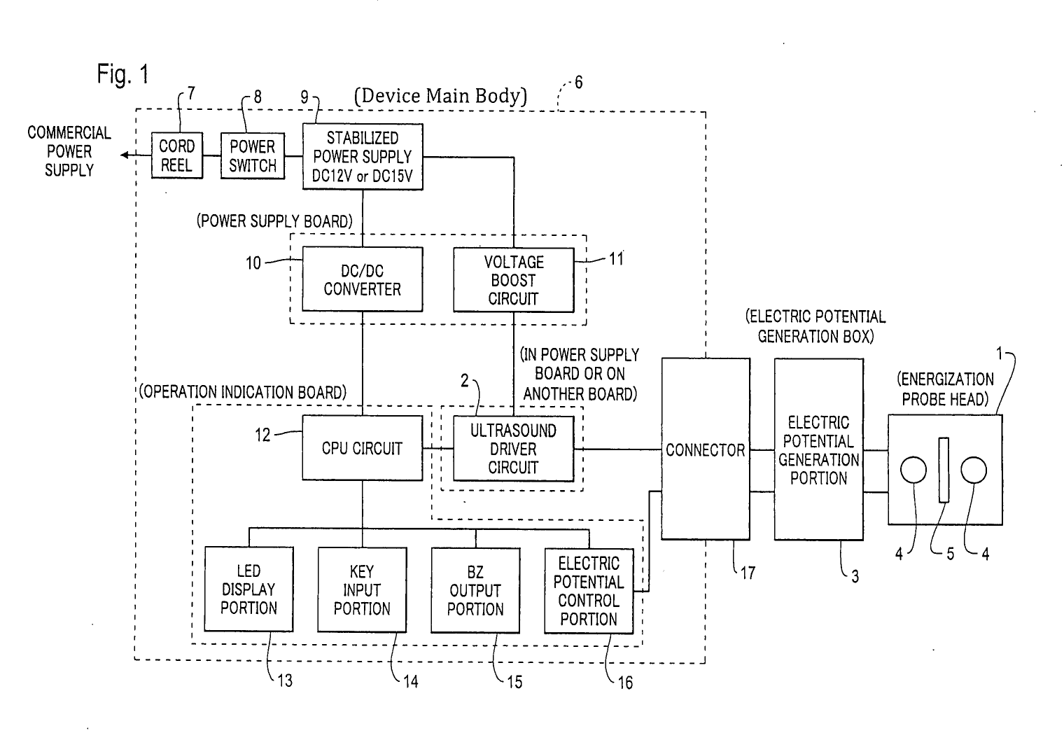

An ultrasound constipation relief device according to the present invention includes, as illustrated in FIG. 1, a head (energization probe head) 1, an ultrasound signal oscillation circuit (ultrasound driver circuit) 2, and an electric potential generation portion 3.

The head 1 is placeable on the back, and generates ultrasound to be transmitted to the lumbar spine. The head of the ultrasound constipation relief device according to the present invention may be placeable on any portion of the lumbar spine, but is desired to be placeable on the lumbar spines L₃ to L₅ from outside the back. The head illustrated as an example is placeable between the lumbar spines L₃ and L₄ or between the lumbar spines L₄ and L₅.

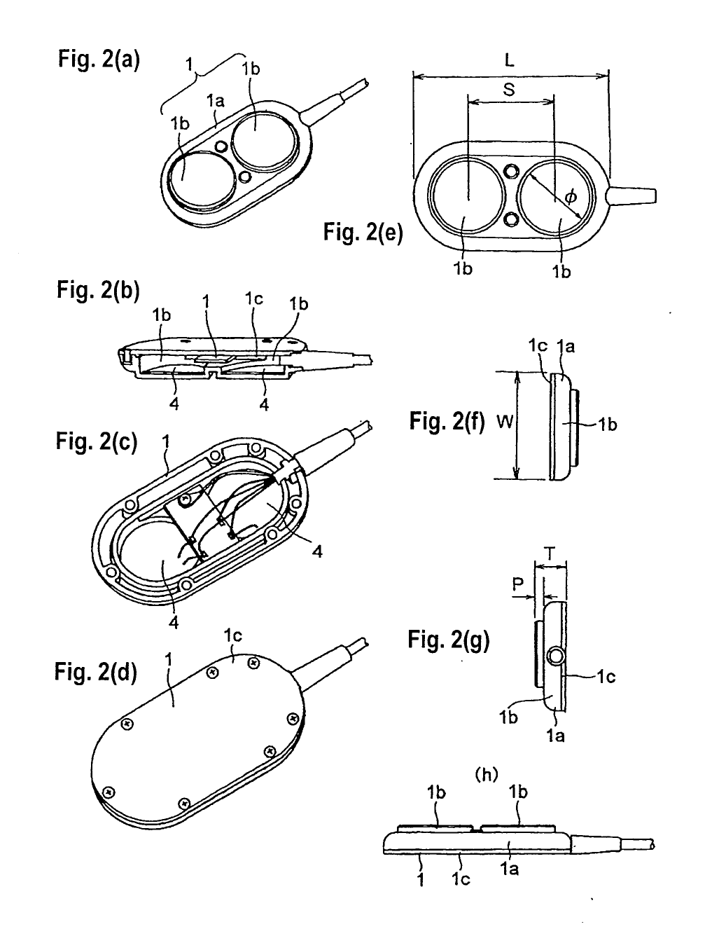

The head 1 of FIGS. 1 and 2 (a) to 2 (h) has two piezoelectric elements 4 built in a head case la made of an insulating resin. The piezoelectric element 4 is an element that converts electric energy into physical vibration when applied with a high frequency signal oscillated from the ultrasound signal oscillation circuit 2. The piezoelectric element 4 has a resonance frequency. In FIG. 1, the two piezoelectric elements 4 have resonance frequencies of 1.5 MHz and 2.0 MHz, respectively. The resonance frequency of the piezoelectric element 4 may be another frequency. Two or more piezoelectric elements 4 may also be used, and the resonance frequencies thereof may be the same or different.

The head case 1a is suitably formed of a hard resin, which easily transmits ultrasound vibration of the piezoelectric elements 4, such as polyacetal (commercial polyacetal: DURACON: product of Polyplastics Co., Ltd.). The head case la is provided with two accommodation portions 1b, which are recessed in a circle, and the piezoelectric elements 4 are accommodated inside the two accommodation portions 1b, respectively. A lid 1c is covered and fixed onto the bottom surface of the head case 1a. The lid is can be made of various kinds of materials, such as polypropylene (PP resin), acrylonitrile butadiene styrene (ABS resin), and polyacetal. In the case where the head case 1a and the lid 1c are made of a soft material, the ultrasound vibration oscillated from the piezoelectric elements 4 is absorbed and less transmitted to the lumbar spine. As the material, a hard material such as metal (including titanium) maybe used. In this case, however, there is a fear that the hard material is heated by the ultrasound vibration and a site having the hard material placed thereon has a low-temperature burn. If design is made so that the heat is prevented from transferring to the back, even a hard material having good conductivity such as metal can be used.

The two accommodation portions 1b protrude over the surface of the head case 1a so that, when the head 1 is to be placed on the back, the positions of the piezoelectric elements 4 may be easily checked and the head 1 may be easily positioned (placed) on the lumbar spine. The shape of the illustrated head case 1a is an ellipse. The size thereof can be selected depending on the use, such as the use for adults or children. As an example, the length L is 80.00±6.00 mm, the width W is 45.00±6.00 mm, the interval S between the center portions of the two accommodation portions 1b is 35.00±5.00 mm, the thickness T is 12.00±3.00 mm, the projection dimension P on the surface side of the accommodation portion 1b is 3.00 mm, and the diameter φ of the accommodation portion 1b is 26.00±6.00 mm. However, other dimensions are possible. It is desired that the head 1 be easy to be placed along with the curve of the back. For this, the head 1 may be made of such structure or material that the head 1 maybe curved between the two accommodation portions 1b. The number of the accommodation portions 1b may be three or more.

The ultrasound signal oscillation circuit 2 is a circuit that generates a signal having a frequency around the resonance frequency of the piezoelectric element 4 provided in the head 1, and applies the signal to the piezoelectric element 4. The piezoelectric element 4 is an element that converts electric energy into physical vibration (ultrasound) when applied with the signal oscillated by the ultrasound signal oscillation circuit 2. As the piezoelectric element 4, an existing piezoelectric element can be used. An oscillation frequency of the ultrasound signal oscillation circuit 2 is determined by the resonance frequency of the piezoelectric element 4. In FIG. 1, the two piezoelectric elements 4 having the resonance frequencies of 1.5 MHz and 2.0 MHz are provided. Accordingly, the oscillation frequency of the ultrasound signal oscillation circuit 2 is set so as to oscillate two signals of 1.5 MHz and 2.0 MHz. The piezoelectric element 4 may have a resonance frequency different from the above-mentioned resonance frequency.

Two ultrasound signal oscillation circuits 2 having different oscillation frequencies are prepared. The two oscillation circuits 2 can be switched automatically by a computer (CPU circuit to be described later) or can be switched manually. As an example of the switching, after the oscillation is performed at an oscillation frequency of 1.5 MHz for 100 μsec, the oscillation is automatically switched to an oscillation frequency of 2.0 MHz for 100 μsec, and the switching is thereafter repeated automatically. The oscillation duration is not limited to 100 μsec and can be switched to another time period. Depending on the situation, the oscillation may be performed at 1.5 MHz and 2.0 MHz simultaneously. The appropriate value of oscillation output power of the ultrasound signal oscillation circuit 2 varies depending on a user's physical constitution, weight, body fat, and the like, but is suitably about several hundred mW to about 800 mW as an example. Such conditions can be set by input from a KEY input portion 14 to be described later. Respective setting of the device is performed, and an operation of starting/stopping the operation is performed. An input signal is controlled by the CPU circuit 12.

The electric potential generation portion 3 includes a circuit for generating a high voltage (low voltage=AC 150 V, high voltage=AC 300 V, 40 kHz to 70 kHz), and applies the generated voltage to an electric potential energization electrode 5 which is built in the head case 1a of the head 1. The generation voltage of the electric potential generation portion 3 may also be another necessary voltage. The electric potential energization electrode 5 applies the high voltage generated by the electric potential generation portion 3, and has a structure insulated by the head case 1a.

The ultrasound signal oscillation circuit 2 is built in the device main body (casing) 6. The device main body 6 includes a cord reel 7, a power switch 8, a stabilized power supply 9, a DC/DC converter 10, a voltage boost circuit 11, a CPU circuit 12, an LED display portion 13, a KEY input portion 14, a buzzer (BZ) output portion 15, an electric potential control portion 16, and a connector 17.

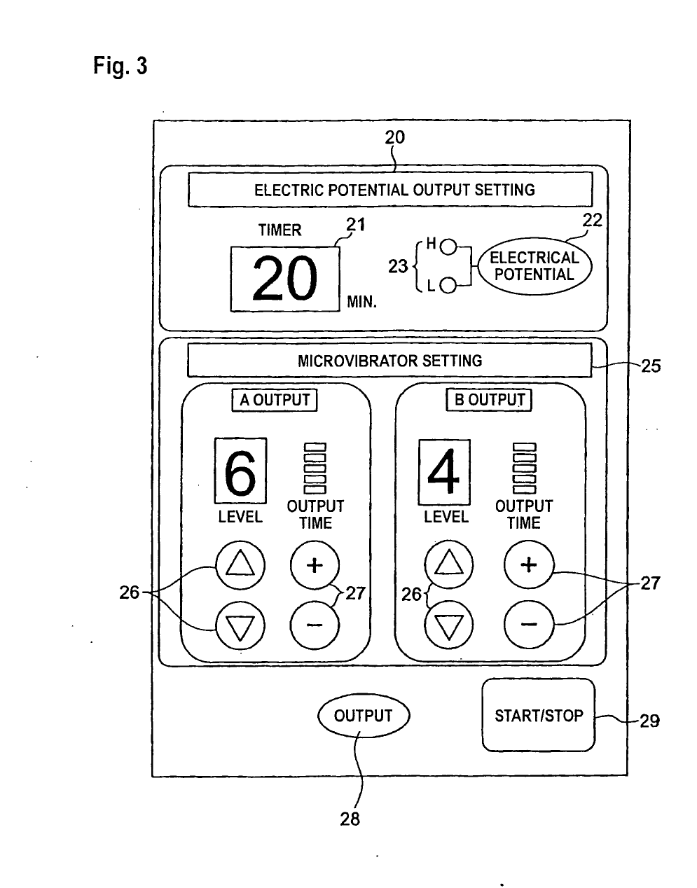

As illustrated in FIG. 3, the device main body 6 is provided with, on an electric potential output setting portion 20, a time indicating portion (LED display) of timer 21, an electric potential voltage changeover portion switch 22, and an electric potential level indicator lamp 23. As illustrated in FIG. 3, the device main body 6 includes a microvibrator (ultrasound output) setting portion 25, and includes an A output portion capable of switching one of the outputs of the two piezoelectric elements 4 and a B output portion capable of switching the other output. At each of the output portions, a level switch button 26 and an output time adjustable button 27 are provided. Further, an operation indicator lamp 28 and a start/stop switch 29 are provided.

The cord reel 7 of FIG. 1 has a power supply cord for supplying power to the device from the commercial power supply, which is reeled so as to be drawable.

The power switch 8 of FIG. 1 has two poles and is capable of opening/closing two circuits, for turning ON/OFF of power supply to the device.

The stabilized power supply 9 of FIG. 1 is a power supply device for converting an input voltage of a commercial power supply (typically AC 85 V to AC 264 V) into DC 12 V (or DC 15 V) and supplying the converted voltage. The input voltage may be converted into another DC voltage.

The DC/DC converter 10 of FIG. 1 is a DC/DC converter circuit (or a series regulator circuit) for stepping down the voltage of DC 12 V (or DC 15 V) supplied from the stabilized power supply 9 to DC 5 V (may be another voltage) for driving the CPU circuit.

The voltage boost circuit 11 of FIG. 1 generates a power supply for the ultrasound signal oscillation circuit 2. The voltage thereof is assumed to be DC 24 V, but is finally determined by output energy of ultrasound.

The CPU circuit 12 of FIG. 1 is a microcomputer circuit for controlling the overall function of the device. The CPU circuit 12 controls all the LED display portion 13, the KEY input portion 14, the BZ output portion 15, and the like, thereby performing BZ beeping, ultrasound output, electric potential output, time counting, and the like.

The LED display portion 13 of FIG. 1 is a portion serving as a monitor for displaying the operation status of the device, and is controlled by the CPU circuit 12.

The KEY input portion 14 of FIG. 1 is a portion for performing input setting of the respective portions of the device, and performing an operation of starting/stopping the operation. An input signal is controlled by the CPU circuit 12.

The BZ output portion 15 of FIG. 1 emits a KEY operation sound at the generation of the input signal of the KEY input portion 14, and an ending sound at the end of the operation. Beeping is controlled by the CPU circuit 12.

The electric potential control portion 16 of FIG. 1 is a circuit for controlling a voltage to be input to the electric potential generation portion 3, and includes a circuit for generating a high voltage (low=AC 150 V, high=AC 300 V, 40 kHz to 70 kHz). The voltage generated by the electric potential control portion 16 is input to the electric potential generation portion 3, and the voltage to be output from the electric potential generation portion 3 is switched between low and high depending on the level of the input voltage. The voltage generated by the electric potential generation portion 3 is applied to the electric potential energization electrode 5.

The connector 17 of FIG. 1 is a coupler that removably connects the device main body 6 and the electric potential generation portion 3. The connector 17 may be a coupler that removably connects the head 1, and may be a coupler that removably connects both the electric potential generation portion 3 and the head 1.

(Usage Example of Ultrasound Constipation Relief Device)

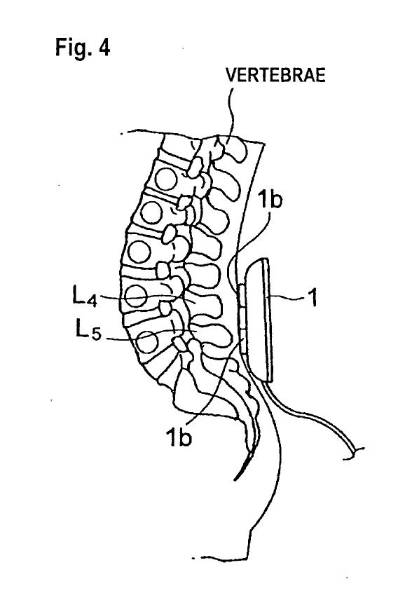

As an example of the usage of the ultrasound constipation relief device according to the present invention, as illustrated in FIG. 4, the head 1 is placed on the back of a person who is seated in a chair, and is fixed onto the back with the use of adhesive tape, an adhesive, a belt, or the like. At this time, the portion of the piezoelectric elements 4 of the head 1 is positioned at the positions of the lumbar spines L₃ to L₅. In this state, an ultrasound signal is oscillated from the ultrasound signal oscillation circuit 2 to be applied to the piezoelectric element 4. The piezoelectric elements 4 accordingly vibrate to generate ultrasound (vibration), which is transmitted to the lumbar spines L₃ to L₅ of the body and stimulates the vicinity thereof, thereby relieving constipation. The usage duration (ultrasound) varies depending on a user, but is suitable to be about several tens of minutes per use. Days for use are variable depending on a user's physical condition, for example, every day or every several days.

An example of the method of fixing the head 1 onto the back is that a user lies on his/her stomach, the head 1 is placed on the back of the user, and a weight such as a sandbag is placed on the head 1 to fix the head 1. Alternatively, a user may lie on his/her back so that the head 1 is laid under the back.

In the case where the ultrasound constipation relief device according to the present invention is used as an electric potential therapy device, for example, the head 1 is placed on the back corresponding to the lumbar spines L₃ to L₅, and the electric potential generation portion 3 is controlled by the electric potential control portion 16, to thereby generate a high voltage (“low”=AC 150 V, “high”=AC 300 V, 40 kHz to 70 kHz) from the electric potential generation portion 3. When the high voltage is applied to the electric potential energization electrode 5 to generate an electric field (to apply an electric field to a human body), the voltage is applied to the human body having the head 1 placed thereon, and a microcurrent (several mA) flows therethrough, thereby performing electric potential therapy.

(Experiment on the Use of the Ultrasound Constipation Relief Device)

An experiment was performed on 4 male subjects and 21 female subjects having the ultrasound constipation relief device according to the present invention applied thereto. The results are shown in Table 1. The age of each subject, the date of the experiment, and the condition of defecation (constipation) before the use of the ultrasound constipation relief device according to the present invention are as shown in Table 1. In this experiment, each subject lay on his/her stomach, and the head 1 of the ultrasound constipation relief device according to the present invention was placed on the back. At this time, the portion of the piezoelectric element 4 of the head 1 was placed on the region between the lumbar spines L₄ and L₅ or the vicinity thereof, and a sandbag (weight) was placed on the head 1 to fix the head 1. In this state, ultrasound generated from the piezoelectric element 4, which had a frequency of 1.5 MHz (2.0 MHz in part) and output power of 100 mW, was transmitted from outside the back to the region between the lumbar spines L₄ and L₅ or the vicinity thereof. One experiment time per person is 20 minutes.

The results of Table 1 are the results within 24 hours after the start of the experiment. As seen from Table 1, the total number of the experiments on 25 subjects was 146. Of 146 experiments, defecation appeared in 84 experiments, being more than half. No defecation appeared in 62 experiments, being less than half. Time before defecation after the start of the experiment was about 1 hour at the earliest and about 22 hours at the latest. As is apparent from the experiment results of Table 1, with the use of the ultrasound constipation relief device according to the present invention, the constipation relieving effect was observed in more than half of the experiments. Although the data was not organized, the constipation relieving effect was improved when the output power was increased (for example, 300 to 500 mW), but, if too strong, some subjects had a touch of diarrhea.

TABLE 1

| | | | Defecation | | | Pulse | | | | |
|---|---|---|---|---|---|---|---|---|---|---|
| Subject | | | frequency | | | fre- | Time for | Time | | |
| | | gen- | before test | | Date | quency | applica- | before | | |
| No. | age | der | (Once/days) | | (2010) | (MHz) | tion | defecation | | Remark |
| 1 | 80 | M | 1/1 | day | 1/6 | 1.5 | 11:30 | x | | Griping discomfort after application, improved condition |
| | | | | | | | | | | due to sudden stop of gas generation. |
| 2 | 76 | F | 1/2 | days | 4/15 | 1.5 | 9:30 | x | | No response after application. |
| | | | | | 4/21 | 1.5 | 9:55 | 5 | hours | |
| | | | | | | | | 05 | minuts | |
| 3 | 78 | F | 1/2-3 | days | 3/5 | 1.5 | 8:50 | x | | No response after application. The subsequent defecation |
| | | | | | | | | | | desire, no defecation for 24 hours. |
| | | | | | 3/7 | 1.5 | 9:00 | x | | No response after application. |
| | | | | | 3/16 | 1.5 | 9:00 | x | | No response after application. |
| 4 | 73 | F | 1/3 | days | 1/6 | 1.5 | 9:30 | 11 h. | 30 m. | Feeling of distension in the abdomen during application. |
| | | | | | 1/7 | 1.5 | 9:15 | x | | No response after application. |
| | | | | | 1/8 | 2.0 | 9:30 | 11 | hours | Feeling of distension in the abdomen after application. |
| | | | | | | | | | | Start of gas generation around 19:00. |
| | | | | | 1/13 | 1.5 | 10:00 | 9 | hours | Griping discomfort after application, defecation at 8:00 |
| | | | | | | | | | | on the next day. |
| | | | | | 1/14 | 1.5 | 15:40 | 16 h. | 20 m. | Feeling of distension in the abdomen after application. |
| | | | | | 1/15 | 1.5 | 11:15 | 8 h. | 45 m. | Defecation at 7:30 in the next morning. |
| | | | | | 1/16 | 1.5 | 10:10 | 21 h. | 10 m. | Feeling of distension in the abdomen after application. |
| | | | | | 1/17 | 1.5 | 9:35 | 9 h. | 55 m. | No response after application. |
| | | | | | 1/19 | 1.5 | 15:15 | 16 h. | 15 m. | Feeling of warmth in the abdomen after application. |
| | | | | | 1/20 | 1.5 | 2:40 | 4 h. | 50 m. | Slight feeling of distention in the abdomen after application. |
| | | | | | 1/26 | 1.5 | 9:10 | 22 h. | 20 m. | No response during application. |
| | | | | | 1/27 | 1.5 | 9:30 | 22 | hours | No response during application. |
| | | | | | 1/28 | 1.5 | 10:15 | x | | Feeling of distention in the abdomen after application. |
| | | | | | 2/3 | 1.5 | 16:00 | x | | Feeling of warmth in the abdomen after application. Feeling |
| | | | | | | | | | | of distention in the abdomen, no defecation for 24 hours. |
| | | | | | 2/4 | 2.0 | 9:50 | 21 h. | 40 m. | Feeling of warmth in the abdomen after application. |
| | | | | | 2/9 | 1.5 | 11:00 | 20 | hours | No response during application. |
| | | | | | 2/10 | 1.5 | 10:30 | 20 | hours | Distention in the abdomen after application. |
| | | | | | 2/12 | 1.5 | 14:55 | 15 h. | 35 m. | Slight feeling of distention in the abdomen after application. |
| | | | | | 2/13 | 1.5 | 10:05 | 22 h. | 25 m. | No response during application. |
| | | | | | 2/17 | 1.5 | 10:35 | x | | Distention in the abdomen after application, no defecation. |
| | | | | | 2/19 | 2.0 | 15:15 | 16 h. | 45 m. | Distention in the abdomen after application. |
| | | | | | 2/21 | 2.0 | 10:40 | x | | Feeling of warmth in the abdomen after application. |
| | | | | | 2/24 | 1.5 | 11:20 | x | | Distention in the abdomen after application, no defecation. |
| | | | | | 3/6 | 1.5 | 9:25 | x | | No response after application. |
| | | | | | 3/12 | 1.5 | 9:20 | x | | No response after application. |
| | | | | | 3/28 | 1.5 | 10:45 | 1 h. | 15 m. | Feeling of warmth in the abdomen after applica- |
| | | | | | | | | | | tion→defecation around noon. |
| | | | | | 3/31 | 1.5 | 11:00 | 20 | hours | Distention in the abdomen after application → defecation |
| | | | | | | | | | | around 7:00 on the next day. |
| | | | | | 4/1 | 1.5 | 11:00 | x | | No response after application. |
| | | | | | 4/2 | 1.5 | 9:00 | x | | |
| | | | | | 4/7 | 1.5 | 14:30 | 17 h. | 30 m. | No response after application. → Defecation around 08:00 |
| | | | | | | | | | | on the next day. |
| | | | | | 4/8 | 1.5 | 9:40 | x | | No response after application → no response for 24 hours. |
| | | | | | | | | | | Application at 2.0 MHz next time. |
| | | | | | 4/13 | 2.0 | 11:40 | 19 h. | 50 m. | “Griping” discomfort in the abdomen after application → |
| | | | | | | | | | | defecation around 7:30 on the next day. |
| | | | | | 4/14 | 2.0 | 9:45 | 21 h. | 15 m. | No response after application → defecation around 7:00 |
| | | | | | | | | | | on the next day. |
| | | | | | 4/16 | 2.0 | 9:45 | x | | No response after application. |
| | | | | | 4/20 | 2.0 | 15:00 | x | | |
| | | | | | 4/27 | 1.5 | 9:50 | x | | No response after application. |
| 5 | 58 | F | 1/4 | days | 1/8 | 1.5 | 11:25 | x | | No response. |
| 6 | 73 | F | 1/2 | days | 4/23 | 1.5 | 14:30 | 30 | minutes | No response after application→surprise at a “quick response.” |
| | | | | | 4/24 | 1.5 | 10:20 | 5 h. | 40 m. | No response after application→desire to stop the application |
| | | | | | | | | | | for a while because of excess defecation. |
| 7 | 84 | F | No | | 1/7 | 1.5 | 16:00 | x | | No response after application, no symptom during the |
| | | | defecation | | | | | | | subsequent period of time. |
| | | | without | | 1/14 | 1.5 | 14:30 | Time | | Appreciation made shaped stool for the first time in |
| | | | laxative | | | | | unknown | | several years. |
| | | | | | 2/7 | 1.5 | 10:45 | x | | No response. |
| 8 | 89 | F | 1/3 | days | 1/20 | 1.5 | 8:55 | 4 h. | 35 m. | No response after application. |
| 9 | 78 | F | 1/2 | days | 1/21 | 1.5 | 9:00 | 27 | hours | Abdominal gurgling, defecation around noon. |
| 10 | 73 | F | 1/3 | days | 1/5 | 1.5 | 11:40 | 1 h. | 5 m. | No response during application. |
| | | | | | 1/7 | 1.5 | 14:00 | x | | No response. |
| | | | | | 1/20 | 1.5 | 16:00 | 16 h. | 10 m. | No response during application. |
| | | | | | 1/21 | 1.5 | 9:25 | x | | Feeling of distention in the abdomen after application. |
| | | | | | 1/21 | 1.5 | 16:30 | x | | No response. |
| | | | | | 1/23 | 2.0 | 11:45 | x | | No response. |
| | | | | | 1/24 | 1.5 | 10:30 | 9 h. | 30 m. | Defecation. |
| | | | | | 1/26 | 2.0 | 11:20 | 6 h. | 40 m. | No response during application. |
| | | | | | 1/27 | 2.0 | 11:20 | 1 h. | 10 m. | No response during application. |
| | | | | | 2/12 | 2.0 | 11:15 | x | | No response. |
| | | | | | 2/23 | 1.5 | 10:50 | x | | No response. |
| | | | | | 3/7 | 1.5 | 10:00 | x | | No response after application. |
| 11 | 82 | F | 1/2 | days | 2/19 | 1.5 | 9:10 | x | | Feeling of warmth in the abdomen after application. |
| | | | | | | | | | | Defecation around 14:00 two days after the application. As |
| | | | | | | | | | | usual. |
| | | | | | 2/26 | 2.0 | 9:20 | 13 h. | 40 m. | No response after application. |
| | | | | | 3/5 | 1.5 | 9:15 | 1 h. | 45 m. | Defecation on the next day. |
| | | | | | 3/12 | 1.5 | 8:50 | x | | No response after application. |
| 12 | 66 | F | 1/3-4 | days | 1/5 | 1.5 | 11:20 | 21 h. | 40 m. | No response during application. |
| | | | | | 1/6 | 1.5 | 11:00 | 21 h. | 30 m. | No response during application, feeling of bowel movement |
| | | | | | | | | | | around 21:00. |
| 13 | 67 | F | 1/3 | days | 2/13 | 1.5 | 9:00 | x | | No response. |
| 14 | 73 | F | 1/7-10 | days | 3/9 | 1.5 | 9:00 | x | | No response after application. |
| 15 | 78 | F | 1/1 day in | | 1/21 | 1.5 | 10:40 | 20 h. | 20 m. | Gurgling discomfort in the abdomen during application. |
| | | | small amount | | 2/10 | 1.5 | 11:10 | x | | No response. |
| | | | | | 4/23 | 1.5 | 14:50 | x | | No response after application. |
| 16 | 72 | M | 1/7 | days | 2/12 | 1.5 | 15:20 | 22 h. | 10 m. | No defecation for one week in spite of taking a purgative. |
| | | | | | | | | | | Defecation on the next day after application. |
| | | | | | 2/14 | 1.5 | 9:10 | x | | No response. |
| 17 | 71 | F | 1/2 | days | 1/5 | 1.5 | 10:30 | 10 | hours | No response during application. |
| | | | | | 1/6 | 1.5 | 10:00 | 3 | hours | Gurgling discomfort in the abdomen during application. |
| | | | | | 1/17 | 1.5 | 10:00 | x | | Slight feeling of distention in the abdomen after application. |
| | | | | | 1/19 | 2.0 | 10:25 | x | | No response. |
| | | | | | 1/20 | 2.0 | 10:30 | x | | No response. |
| 18 | 81 | F | 1/3 | days | 2/3 | 1.5 | 15:15 | 16 h. | 5 m. | No response after application. |
| 19 | 75 | M | 1/1 | day | 2/6 | 1.5 | 15:00 | x | | No response. |
| 20 | 70 | F | Nearly 1/1 | | 1/14 | 1.5 | 10:15 | 2 h. | 55 m. | Larger amount of stool than usual. |
| | | | | | 1/15 | 1.5 | 10:20 | 19 h. | 40 m. | Feeling of distention in the abdomen five minutes after |
| | | | | | | | | | | application. Defecation in the morning, small amount of stool |
| | | | | | | | | | | in separate hard lumps. |
| | | | | | 1/17 | 2.0 | 11:40 | 1 h. | 50 m. | Feeling of warmth around the applied part in the lumbar |
| | | | | | | | | | | region after application. No skin abnormality such as scald. |
| | | | | | | | | | | Larger amount of stool than usual. |
| | | | | | 1/19 | 1.5 | 10:00 | 6 | hours | Small amount of stool. |
| | | | | | 1/20 | 1.5 | 10:00 | 6 | hours | Defecation (small amount) three times from 16:00 |
| | | | | | 1/21 | 1.5 | 10:00 | 4 | hours | Small amount of stool. “Defecation is becoming easier.” |
| | | | | | 1/22 | 1.5 | 10:15 | 3 h. | 45 m. | Small amount of stool, the same amount as usual. |
| | | | | | 1/23 | 1.5 | 9:55 | 3 h. | 35 m. | Small amount of stool, the same amount as usual. |
| | | | | | 1/27 | 2.0 | 10:00 | 3 h. | 30 m. | Feeling of warmth around the applied part in the lumbar |
| | | | | | | | | | | region after application. No skin abnormality such as scald. |
| | | | | | | | | | | The same amount of stool as usual. |
| | | | | | 1/28 | 2.0 | 9:50 | x | | No response. |
| | | | | | 2/3 | 2.0 | 9:45 | 3 h. | 15 m. | Small amount of stool in separate hard lumps. “Defecation |
| | | | | | | | | | | was easier than usual.” |
| | | | | | 2/4 | 2.0 | 10:45 | 5 h. | 15 m. | The same amount of stool as usual. |
| | | | | | 2/6 | 2.0 | 10:50 | 2 h. | 10 m. | Small amount of stool, easier defecation than usual. |
| | | | | | 2/7 | 2.0 | 10:15 | 2 h. | 45 m. | Defecation from around 13:00. Larger amount and frequency |
| | | | | | | | | | | of stool than usual. |
| | | | | | 2/9 | 2.0 | 9:30 | 1 | hour | Abdominal gurgling, defecation (small amount) around 10:30. |
| | | | | | | | | | | Defecation (large amount) around 6:00 on in the next morning. |
| | | | | | 2/10 | 2.0 | 9:30 | 2 | hours | Small amount of stool, easier defecation than usual. |
| | | | | | 2/12 | 2.0 | 9:30 | 4 h. | 30 m. | Gurgling discomfort in the abdomen after application. Larger |
| | | | | | | | | | | amount and better shape of stool than usual. Defecation |
| | | | | | | | | | | three times during the subsequent period of time. |
| | | | | | 2/13 | 2.0 | 10:30 | 3 h. | 30 m. | Gurgling discomfort in the abdomen after application. |
| | | | | | 2/16 | 2.0 | 10:00 | x | | No response. |
| | | | | | 2/17 | 2.0 | 10:05 | 23 h. | 55 m. | “There was defecation, which was not easy.” |
| | | | | | 2/20 | 2.0 | 10:00 | 3 | hours | Larger amount and better shape of stool than usual. |
| | | | | | 2/21 | 2.0 | 10:15 | 4 h. | 45 m. | Small amount of stool. |
| | | | | | 3/7 | 1.5 | 10:30 | x | | Discomfort in the abdomen after application → defecation. |
| | | | | | 3/9 | 1.5 | 15:00 | x | | No response after application → no defecation. Application |
| | | | | | | | | | | at 2.0 MHz next time. |
| | | | | | 3/10 | 2.0 | 10:40 | 2 h. | 50 m. | No response after application → defecation. |
| | | | | | 3/11 | 2.0 | 10:00 | x | | No response after application → no response for 24 hours. |
| | | | | | 3/14 | 2.0 | 10:00 | x | | No response after application. |
| | | | | | 3/16 | 1.5 | 10:35 | 5 h. | 25 m. | No response after application → defecation (small amount). |
| | | | | | 3/17 | 1.5 | 11:55 | 1 h. | 35 m. | Defecation around 13:30 to 14:30 |
| | | | | | 3/18 | 1.5 | 15:35 | 25 | m. | No response after application → defecation (small amount). |
| | | | | | 3/19 | 1.5 | 8:50 | x | | No response after application. |
| | | | | | 3/23 | 1.5 | 9:50 | 6 h. | 10 m. | No response after application → defecation (small amount). |
| | | | | | 3/24 | 1.5 | 11:10 | 1 h. | 50 m. | |
| | | | | | 3/25 | 1.5 | 11:00 | x | | No response after application. |
| | | | | | 4/7 | 1.5 | 10:30 | x | | No response after application → no response for 24 hours. |
| | | | | | 4/8 | 1.5 | 10:10 | x | | No response after application → no response for 24 hours. |
| | | | | | | | | | | Application at 2.0 MHz next time. |
| | | | | | 4/10 | 2.0 | 9:55 | 3 h. | 35 m. | No response after application → defecation twice around |
| | | | | | | | | | | 13:30 to 15:30. |
| | | | | | 4/11 | 2.0 | 10:25 | x | | No response after application → defecation within 24 hours. |
| | | | | | 4/14 | 2.0 | 10:30 | x | | No response after application→defecation twice between |
| | | | | | | | | | | 14:00 and 16:00. Small amount of stool in “separate hard |
| | | | | | | | | | | lumps.” Feeling that defecation was made easier through |
| | | | | | | | | | | the use of a device. |
| | | | | | 4/16 | 2.0 | 10:10 | 4 h. | 50 m. | No response after application → defecation twice around 15:00 |
| | | | | | | | | | | to 16:30. |
| | | | | | 4/20 | 2.0 | 9:30 | 5 h. | 30 m. | No response after application → 15:00 defecation |
| | | | | | | | | | | around 08:00. |
| | | | | | 4/21 | 2.0 | 10:35 | x | | No response after application. |
| | | | | | 5/2 | 1.5 | 9:50 | x | | No response after application. |
| 21 | 85 | F | 1/1 | day | 2/9 | 1.5 | 9:00 | 22 h. | 30 m. | Defecation in the next morning. “Feeling that defecation |
| | | | | | | | | | | was easier than usual.” |
| | | | | | 2/18 | 1.5 | 9:10 | x | | No response. |
| 22 | 74 | F | 1/1 | day | 2/4 | 1.5 | 9:20 | 11 h. | 40 m. | Gradual increase in pain in the abdomen. Diarrhea-like |
| | | | | | | | | | | response around 21:00. |
| 23 | 73 | F | 1/3 | days | 4/7 | 1.5 | 16:35 | 14 h. | 25 m. | No response after application → defecation around 7:00 on |
| | | | | | | | | | | the next day. |
| | | | | | 4/8 | 1.5 | 10:40 | 20 h. | 20 m. | No response after application → defecation around 7:00 on |
| | | | | | | | | | | the next day . |
| | | | | | 4/9 | 1.5 | 9:30 | 21 | hours | No response after application → defecation around 6:30 on |
| | | | | | | | | | | the next day. |
| | | | | | 4/10 | 1.5 | 9:30 | 21 | hours | No response after application → defecation around 6:30 on |
| | | | | | | | | | | the next day. |
| | | | | | 4/11 | 1.5 | 9:40 | 21 h. | 20 m. | No response after application → defecation in the morning |
| | | | | | | | | | | on the next day. |
| | | | | | 4/13 | 1.5 | 9:45 | 21 h. | 15 m. | No response after application → defecation in the morning |
| | | | | | | | | | | on the next day. |
| | | | | | 4/16 | 1.5 | 11:00 | 19 h. | 30 m. | No response after application → defecation around 6:30 on |
| | | | | | | | | | | the next day. |
| | | | | | 4/17 | 1.5 | 14:30 | 16 | hours | No response after application → defecation around 6:30 on |
| | | | | | | | | | | the next day. |
| | | | | | 4/18 | 1.5 | 10:40 | x | | No response after application |
| | | | | | 4/21 | 1.5 | 11:00 | 20 | hours | No response after application → defecation in the morning |
| | | | | | | | | | | on the next day. |
| | | | | | 4/22 | 1.5 | 9:20 | x | | No response after application. |
| | | | | | 4/25 | 1.5 | 9:30 | x | | No response after application. |
| | | | | | 4/28 | 1.5 | 14:15 | x | | No response after application. |
| | | | | | 5/1 | 1.5 | 10:45 | 20 h. | 15 m. | No response after application → defecation in the morning |
| | | | | | | | | | | on the next day. |
| | | | | | 5/2 | 1.5 | 9:20 | x | | No response after application. |
| 24 | 81 | M | 1/3 | days | 1/7 | 1.5 | 11:15 | x | | Feeling of bowel movement in the abdomen after application. |
| 25 | 71 | F | 1/4 | days | 1/7 | 1.5 | 15:10 | 3 h. | 50 m. | No response during application. Defecation at 8:30 in the |
| | | | | | | | | | | next morning. |

| | | | | |
|---|---|---|---|---|
| 1/3 | 1.5 | 15:15 | x | No response. |

### Reference Signs List

1 head (energization probe head)
1a head case
1b accommodation portion
1c lid
2 ultrasound signal oscillation circuit (ultrasound driver circuit)
3 electric potential generation portion
4 piezoelectric element
5 electric potential energization electrode
6 device main body (casing)
7 cord reel
8 power switch
9 stabilized power supply
10 DC/DC converter
11 voltage boost circuit
12 CPU circuit
13 LED display portion
14 KEY input portion
15 buzzer (BZ) output portion
16 electric potential control portion
17 connector
20 electric potential output setting portion
21 time indicating portion (LED display) of timer
22 electric potential voltage changeover portion switch
23 electric potential level indicator lamp
25 microvibrator (ultrasound output) setting portion
26 level switch button
27 output time adjustable button
28 operation indicator lamp
29 start/stop switch

1. An ultrasound constipation relief device, which is capable of relieving constipation or inducing defecation by transmitting ultrasound vibration from a back to both or any one of a lumbar spine and a region between lumbar spines so as to stimulate a pressure point or a nerve relating to constipation,
the ultrasound constipation relief device comprising:
an ultrasound signal oscillation circuit;
a single head, which is placeable from the back on at least a region between a third lumbar spine and a fourth lumbar spine, a region between the fourth lumbar spine and a fifth lumbar spine, or any one or two or more of the third lumbar spine, the fourth lumbar spine, and the fifth lumbar spine; and
a piezoelectric element, which is to be vibrated by an ultrasound signal oscillated from the ultrasound signal oscillation circuit to generate ultrasound capable of being transmitted from outside the back to the lumbar spine or the region between lumbar spines,
wherein the piezoelectric element is fixed onto the single head made of a hard material which easily transmits the ultrasound vibration generated from the piezoelectric element,
wherein the piezoelectric element is capable of generating ultrasound vibration having a frequency of 1.5 MHz to 2.0 MHz,
wherein the single head has a size which is placeable on the region between the third lumbar spine and the fourth lumbar spine, the region between the fourth lumbar spine and the fifth lumbar spine, or the any one or two or more of the third lumbar spine, the fourth lumbar spine, and the fifth lumbar spine, and
wherein the single head is capable of outputting the ultrasound vibration having the frequency and output power capable of stimulating the pressure point or the nerve relating to constipation.

2. An ultrasound constipation relief device, which is capable of relieving constipation or inducing defecation by transmitting ultrasound vibration from a back to both or any one of a lumbar spine and a region between lumbar spines so as to stimulate a pressure point or a nerve relating to constipation,
the ultrasound constipation relief device comprising:
an ultrasound signal oscillation circuit;
two or more heads, which are placeable from outside the back on at least a region between a third lumbar spine and a fourth lumbar spine, a region between the fourth lumbar spine and a fifth lumbar spine, or any one or two or more of the third lumbar spine, the fourth lumbar spine, and the fifth lumbar spine; and
a piezoelectric element, which is to be vibrated by an ultrasound signal oscillated from the ultrasound signal oscillation circuit to generate ultrasound capable of being transmitted from outside the back to the lumbar spine or the region between lumbar spines,
wherein the piezoelectric element is provided to each of the two or more heads,
wherein the piezoelectric element is capable of generating ultrasound having a frequency of 1.5 MHz to 2.0 MHz,
wherein the two or more heads are arrayed at an interval in a line or substantially in a line,
wherein the interval of the two or more heads is set so that the two or more heads are placeable on the region between the third lumbar spine and the fourth lumbar spine, the region between the fourth lumbar spine and the fifth lumbar spine, or the any two or more of the third lumbar spine, the fourth lumbar spine, and the fifth lumbar spine, and
wherein the two or more heads are made of a hard material which easily transmits the ultrasound vibration of the piezoelectric element.

3. An ultrasound constipation relief device according to claim 2, wherein the piezoelectric elements of the two or more heads, which are provided at an interval, generate the ultrasound simultaneously or alternately.

4. An ultrasound constipation relief device according to claim 1, wherein the output power of the ultrasound generated from the piezoelectric element is 100 mW to 800 mW.

5. An ultrasound constipation relief device according to claim 1, further comprising:
a high-voltage generation portion; and
an electric potential energization electrode, which is supplied with a high voltage from the high-voltage generation portion,
wherein the electric potential energization electrode is capable of applying the high voltage to a human body so that a microcurrent flows through the human body.

6. An ultrasound constipation relief device according to claim 4, further comprising:
a high-voltage generation portion; and
an electric potential energization electrode, which is supplied with a high voltage from the high-voltage generation portion,
wherein the electric potential energization electrode is capable of applying the high voltage to a human body so that a microcurrent flows through the human body.

7. An ultrasound constipation relief device according to claim 5, wherein the electric potential energization electrode is provided inside a head case of the ultrasound constipation relief device.

8. An ultrasound constipation relief device according to claim 6, wherein the electric potential energization electrode is provided inside a head case of the ultrasound constipation relief device.

9. An ultrasound constipation relief device according to claim 2, wherein the output power of the ultrasound generated from the piezoelectric element is 100 mW to 800 mW.

10. An ultrasound constipation relief device according to claim 3, wherein the output power of the ultrasound generated from the piezoelectric element is 100 mW to 800 mW.

11. An ultrasound constipation relief device according to claim 2, further comprising:
a high-voltage generation portion; and
an electric potential energization electrode, which is supplied with a high voltage from the high-voltage generation portion,
wherein the electric potential energization electrode is capable of applying the high voltage to a human body so that a microcurrent flows through the human body.

12. An ultrasound constipation relief device according to claim 3, further comprising:
a high-voltage generation portion; and
an electric potential energization electrode, which is supplied with a high voltage from the high-voltage generation portion,
wherein the electric potential energization electrode is capable of applying the high voltage to a human body so that a microcurrent flows through the human body.

13. An ultrasound constipation relief device according to claim 9, further comprising:
a high-voltage generation portion; and
an electric potential energization electrode, which is supplied with a high voltage from the high-voltage generation portion,
wherein the electric potential energization electrode is capable of applying the high voltage to a human body so that a microcurrent flows through the human body.

14. An ultrasound constipation relief device according to claim 10, further comprising:
a high-voltage generation portion; and
an electric potential energization electrode, which is supplied with a high voltage from the high-voltage generation portion,
wherein the electric potential energization electrode is capable of applying the high voltage to a human body so that a microcurrent flows through the human body.

15. An ultrasound constipation relief device according to claim 11, wherein the electric potential energization electrode is provided inside a head case of the ultrasound constipation relief device.

16. An ultrasound constipation relief device according to claim 12, wherein the electric potential energization electrode is provided inside a head case of the ultrasound constipation relief device.

17. An ultrasound constipation relief device according to claim 13, wherein the electric potential energization electrode is provided inside a head case of the ultrasound constipation relief device.

18. An ultrasound constipation relief device according to claim 14, wherein the electric potential energization electrode is provided inside a head case of the ultrasound constipation relief device.
